(19) United States
(12) Patent Application Publication
(10) Pub. No.: US 2014/0276096 A1
(43) Pub. Date: Sep. 18, 2014

(54) SYSTEMS AND METHODS FOR USE IN DIAGNOSING A MEDICAL CONDITION OF A PATIENT

(71) Applicant: Bonutti Research, Inc., Effingham, IL (US)

(72) Inventor: Peter M. Bonutti, Effingham, IL (US)

(21) Appl. No.: 13/838,553

(22) Filed: Mar. 15, 2013

(51) Int. Cl. A61B 5/00

(52) U.S. Cl. CPC A61B 5/7275
USPC 600/476; 600/300; 705/2

(57) ABSTRACT

Systems and method for use in diagnosing a medical condition of a patient are provided. The method includes providing medical condition information, receiving patient data relating to the medical condition information, comparing the received data to a baseline, and determining, by a computing device including a processor, a class of patient based on the received patient data.

### BACKGROUND

The present disclosure relates generally to systems and methods for use in diagnosing a patient, and more specifically to systems and methods for use in diagnosing a medical condition of a patient.

Generally, when a patient encounters a health question or issue relating to a musculoskeletal system discomfort and/or problem that patient will visit a general practitioner to aid in the diagnosis of the patient's specific health issue. Often a general practitioner will order diagnostic studies (e.g., MRI, CT, and X-rays) to determine the cause the patient's discomfort. In some instances, these diagnostic studies are unnecessary for the diagnosis of a patient and such studies may add unnecessary costs to the patient and/or an insurance carrier covering the patient. As such, there is a need for cost effective methods and systems for diagnosing medical conditions of patients.

### BRIEF DESCRIPTION

In one aspect of the present disclosure, a method for diagnosing a medical condition of a patient is provided. The method includes providing medical condition information, receiving patient data relating to the medical condition information, comparing the received data to a baseline, and determining, by a computing device including a processor, a class of patient based on the received patient data.

In one aspect of the present disclosure, one or more non-transitory computer-readable storage media having computer-executable instructions embodied thereon are provided. When executed by a processor, the computer-executable instructions cause the processor to provide medical condition information, receive patient data relating to the medical condition information, compare the received data to a baseline, and determine a class of patient based on the received patient data.

In another aspect of the present disclosure, a method for determining a quality of care score for treatment associated with a patient is provided. The method includes receiving patient data, tracking at least one treatment provided to a patient, monitoring at least one result of the at least one treatment, and determining, by a computing device including a processor, a quality of care score based on the at least one result.

In yet another aspect of the present disclosure, one or more non-transitory computer-readable storage media having computer-executable instructions embodied thereon are provided. When executed by a processor, the computer-executable instructions cause the processor to receive patent data, track at least one treatment provided to a patient, monitor at least one result of the at least one treatment, and determine a quality of care score based on the at least one result.

The features, functions, and advantages that have been discussed can be achieved independently in various embodiments or may be combined in yet other embodiments, further details of which can be seen with reference to the following description and drawings.

### BRIEF DESCRIPTION OF THE DRAWINGS

FIG. 1 is a block diagram of an exemplary computing device.

FIG. 2 illustrates an exemplary electronic diagnostic system using the computing device shown in FIG. 1.

FIG. 3 is an exemplary flowchart of a method of diagnosing a medical condition of a patient using the system shown in FIG. 2.

FIG. 4 is an illustration of a patient using the system shown in FIG. 2.

FIG. 5 is an exemplary flowchart of an entire patient experience using the system shown in FIG. 2.

### DETAILED DESCRIPTION

The subject matter described herein relates to electronically diagnosing a medical condition of a patient. More specifically, the subject matter described herein relates to automatically diagnosing an orthopedic condition in a patient based on information received from a portable computing device (e.g., smartphone). A patient's quality of care and satisfaction with care received are integral to patient treatment and management. This is especially true in musculoskeletal injuries. The subject matter described herein provide methods and systems that can be utilized to assist in the spectrum of quality of patient care and satisfaction as well as provide efficiencies and cost effectiveness in the care. The cost effectiveness of the subject matter described herein can begin at a patient's initial contact to treatment and/or recovery including, but not limited to, diagnostic care, medical/surgical treatment, recovery, follow-up care, rehabilitation, long term follow-up with assessments of patient satisfaction, quality, and long term results. The subject matter described herein can be used in conjunction with patient monitoring equipment. An exemplary monitoring system is provided in U.S. Pat. No. 7,182,738 entitled “Patient Monitoring Apparatus and Method for Orthosis and Other Devices,” to Bonutti et al., the content of which is herein expressly incorporated by reference in its entirety.

The subject matter described herein relates to the overall entire issue of spectrum of care. More specifically, the methods and systems described herein relate to early diagnosis of musculoskeletal care. Once a diagnosis is made, patients are triaged and/or classified into appropriate medical/surgical treatments. After receiving the treatment, rehabilitation and recovery from these treatment programs ensues. The methods and systems described herein provide for a follow-up to obtain a patient's satisfaction (e.g., knee and hip scoring systems, etc.) to long term quality/satisfaction and results of their treatment. This could be from a medical evaluation or the use of pharmaceutical agent for treatment to the results of a medical/surgical treatment. If failure of medical treatment would occur, the patient then would progress into surgical treatment and then progress into rehabilitation and recovery. It would shorten the long term outcomes.

As used herein, an element or step recited in the singular and proceeded with the word “a” or “an” should be understood as not excluding plural elements or steps unless such exclusion is explicitly recited. Furthermore, references to “one embodiment” or the “exemplary embodiment” are not intended to be interpreted as excluding the existence of additional embodiments that also incorporate the recited features. Additionally, the term “orthopedic condition”, as used herein, refers to an irregularity found in a patient's musculoskeletal system (e.g., musculoskeletal disorder).

FIG. 1 is a block diagram of an exemplary computing device 10 that may be used to electronically diagnose a medical condition of a patient. In the exemplary embodiment, computing device 10 includes a memory 16 and a processor 14 that is coupled to memory 16 for executing programmed instructions. Processor 14 may include one or more processing units (e.g., in a multi-core configuration). Computing device 10 is programmable to perform one or more operations described herein by programming memory 16 and/or processor 14. For example, processor 14 may be programmed by encoding an operation as one or more executable instructions and providing the executable instructions in memory 16.

Processor 14 may include, but is not limited to, a general purpose central processing unit (CPU), a microcontroller, a reduced instruction set computer (RISC) processor, an application specific integrated circuit (ASIC), a programmable logic circuit (PLC), and/or any other circuit or processor capable of executing the functions described herein. The methods described herein may be encoded as executable instructions embodied in a computer-readable medium including, without limitation, a storage device and/or a memory device. Such instructions, when executed by processor 14, cause processor 14 to perform at least a portion of the methods described herein. The above examples are exemplary only, and thus are not intended to limit in any way the definition and/or meaning of the term processor.

Memory 16, as described herein, is one or more devices that enable information such as executable instructions and/or other data to be stored and retrieved. Memory 16 may include one or more computer-readable media, such as, without limitation, dynamic random access memory (DRAM), static random access memory (SRAM), a solid state disk, and/or a hard disk. Memory 16 may be configured to store, without limitation, questionnaires, motion patterns, and/or any other type of data suitable for use with the methods and systems described herein.

In the exemplary embodiment, computing device 10 includes a presentation device 18 that is coupled to processor 14. Presentation device 18 outputs by, for example, displaying, printing, and/or otherwise outputting information such as, but not limited to, documents, interfaces, warnings, and/or any other type of data to a user 12. For example, presentation device 18 may include a display adapter (not shown in FIG. 1) that is coupled to a display device, such as a cathode ray tube (CRT), a liquid crystal display (LCD), a light-emitting diode (LED) display, an organic LED (OLED) display, and/or an “electronic ink” display. In some embodiments, presentation device 18 includes more than one display device. In one embodiment, display device is a heads-up display that can be incorporated into and/or on wearable items (e.g., glasses).

In the exemplary embodiment, computing device 10 includes an input device 20 that receives input from user 12. For example, input device 20 may be configured to receive input, selections, and/or any other type of inputs from user 12 suitable for use with the methods and systems described herein. In the exemplary embodiment, input device 20 is coupled to processor 14 and may include, for example, a keyboard, a pointing device, a mouse, a stylus, a touch sensitive panel (e.g., a touch pad or a touch screen), and/or an audio input device. In one embodiment, input device includes at least one sensor 21 configured to capture the movement of a patient including but not limited to, an accelerometer, a goniometer, and a video camera. In the exemplary embodiment, sensors 21 communicate wirelessly with other computing device 10 and/or other sensors 21 using a protocol such as, but not limited to Bluetooth. Alternatively, sensors 21 communicate via any communication method that facilitates diagnosing a medical condition of a patient as described herein including but not limited to a wired connection. Further, in various exemplary embodiments, a touch screen, such as included in an IPAD® tablet, registered trademark of Apple Inc., or similar portable communication device, functions as both presentation device 18 and input device 20.

In the exemplary embodiment, computing device 10 includes one or more communication device 22 coupled to memory 16 and/or processor 14. Communication device 22 is coupled in communication with a device spaced apart from computing device 10, such as another computing device 10. For example, communication device 22 may include, without limitation, a wired network adapter, a wireless network adapter, a Bluetooth adapter, and/or a mobile telecommunications adapter. In at least one embodiment, computing device 10 includes processor 14 and one or more communication devices 22 incorporated into or with processor 14. In some embodiments, communication device 22 may be a network adapter, such as a Bluetooth adapter, and/or another long-range or short-range wireless network adapter. While communication device 22 is illustrated as incorporated with processor 14, it should be appreciated that communication device 22 (or another communication device 22) may be separate from processor 14 and/or engage processor 14. In one embodiment, communication device 22 includes a network adapter (e.g., internal to computing device 10) to communicate with a wide area network (WAN).

Instructions for operating systems and applications are located in a functional form on non-transitory memory 16 for execution by processor 14 to perform one or more of the processes described herein. These instructions in the different embodiments may be embodied on different physical or tangible computer-readable media, such as memory 16 or another memory, such as a computer-readable media 24, which may include, without limitation, a flash drive, CD-ROM, thumb drive, floppy disk, etc. Further, instructions are located in a functional form on non-transitory computer-readable media 24, which may include, without limitation, a flash drive, CD-ROM, thumb drive, floppy disk, etc. Computer-readable media 24 is selectively insertable and/or removable from computing device 10 to permit access to and/or execution by processor 14. In one example, computer-readable media 24 includes an optical or magnetic disc that is inserted or placed into a CD/DVD drive or other device associated with memory 16 and/or processor 14. In some instances, computer-readable media 24 may not be removable.

FIG. 2 illustrates an exemplary electronic diagnostic system 100 for use in diagnosing a medical condition of a patient. In the exemplary embodiment, system 100 includes a host server 102, a plurality of portable communication devices 104, and a workstation 105. Portable communication device 104 may include, without limitation, smartphones, personal digital assistants (PDAs), mobile network devices, and/or mobile handheld devices (e.g., an iPad® device), a heads-up display device, etc. It should be appreciated that each of host server 102, portable communication devices 104, and workstation 105 are exemplary computing devices 10.

In the exemplary embodiment, each portable communication device 104 is coupled to host server 102 through a network 106. In the exemplary embodiment, network 106 is a wide area network (WAN). In other embodiments, network 106 may include, without limitation, the Internet, an intranet, a local area network (LAN), a wide area private network, a wide area public network, a mobile network, a virtual network, and/or another suitable network for communicating data between host server 102, another portable communication device 104, and/or other computing devices.

In the exemplary embodiment, one or more portable communication devices 104 and/or host server 102 are configured to provide a patient application, for amongst other things, transmitting questionnaires relating to a medical condition and transmitting motion data of a patient data between devices 104 and/or host server 102. In the exemplary embodiment, one or more portable communication devices 104 and/or host server 102 are configured to provide an administration application for, amongst other things, receiving questionnaires and patient data transmitted between devices 104 and/or host server 102. The patient application and the administration application may be executed by host server 102 and/or by one or more of portable communication devices 104 to selectively display one or more of the plurality of interfaces at portable communication device 104 to user 12. Further, in at least one embodiment, each of the patient application and the administration application may be executed to provide interfaces for presentation to user 12 at a workstation 105, which includes a computing device 10.

FIG. 3 is an exemplary flowchart of a method 200 of diagnosing a medical condition of a patient using system 100 shown in FIG. 2. In the exemplary embodiment, a patient selects a medical condition for diagnosis such that the selection is received 202 by the patient application. By way of example and not limitation, a patient inquiring about an orthopedic condition (i.e., hip problem) will illustrate method 200. For example, if a patient is experiencing pain or discomfort associated with their hip, the patient would select the hip as a medical condition in need of diagnostics such that the selection would be received 202 by the patient application.

In the exemplary embodiment, after receiving 202 patient input, computing device 10 would provide 204 the patient a questionnaire based on the received 202 patient input. In the exemplary embodiment, the provided 204 questionnaire would ask the patient a plurality of questions relating to the medical condition of the received 202 input. For example, in the case of a hip, the questionnaire asks the patient to select answers to questions such as, but not limited to, the distance the patient can walk without assistance, the distance the patient can walk with assistance, whether or not the patient walks with a limp (e.g., Trendelenburg gait), whether or not the patient has the ability to put on shoes and socks, and if and how far a patient can travel on stairs. Once the patient application receives 206 answers to the provided 204 questionnaire from the patient, the patient application provides 208 a motion pattern to the patient.

In the exemplary embodiment, the motion pattern is provided 208 to the patient based on the received 206 answers. The motion pattern is provided 208 such that computing device 10 can track the motion of a patient to diagnosis a medical condition. In the exemplary embodiment, computing device 10 (i.e., portable computing device 104) and sensors 21 are coupled to a patient to track the motion of a patient. It should be noted that computing device 10 and sensors 21 can be coupled to any portion of a patient in any manner that facilitates diagnosing a patient as described herein, including but not limited to using an adhesive, hook and loop fastener, self-adhering wrap, and/or stick-on material. In one embodiment, sensors 21 are coupled to a patient by an article of clothing such as, but not limited to a band, a glove, a sock, and a shoe. For example, after a patient is provided 208 a motion pattern, appropriate sensors are applied or coupled to the legs and/or hip of a patient to capture motion data of the patient. The patient then begins the motion pattern(s) provided 208 including, but not limited to, putting shoes and socks on, walking on a level surface, walking up stairs, and walking down stairs. The data captured is used to compare against a baseline to determine if an irregularity is found in the captured motion pattern.

In one embodiment, sensors embedded and/or included in a portable computing device, including but not limited to, an accelerometer, a goniometer, a camera, and a microphone are used. In one embodiment, sensors are ingested and/or implanted within a body. In some embodiments, multiple devices are used together to provide more precise data. For example, a smartphone 104 can be used with a tablet 104 to capture different video angles of a patient to provide a 3-dimensional view. In one embodiment, processing of data received from sensors is shared and/or distributed among multiple computing devices (e.g., cloud computing).

In one embodiment, video data of the motion pattern is captured by computing device 10. In such an embodiment, markers and/or sensors may be placed on particular portions of the body to provide volumetric analysis of specific motion patterns. It should be noted that the video data may be captured from the computing device 10 providing 208 the motion pattern or the video data may be captured by one or more computing devices in communication with the computing device 10 providing 208 the motion pattern. In one embodiment, system 100 tracks the pain and/or discomfort of a patient going through the provided 208 motion pattern. In such an embodiment, a user performs the provided 208 motion pattern and tracks or selects when pain and/or discomfort is encountered through input device 20 and/or sensors 21 including, but not limited to, a keyboard input, an audible input, and a touch screen selection.

In the exemplary embodiment, patient data (e.g., motion pattern data) is received 210 by system 100 to determine 212 a diagnostic class for the patient based on the received 210 patient data. In the exemplary embodiment, the determined 212 class has a treatment protocol associated with the class for treating the medical condition for the determined 212 class. In one embodiment, the determined 212 class provides a recommendation for further diagnostic studies and/or a specialist for treatment of the medical condition. In one embodiment, a recommendation may be provided to the patient and/or specialist based on the determined 212 class such as but not limited to, range of motion exercises and supplemental orthopedic correctives (e.g., shoe lifts or shoe inserts).

In the exemplary embodiment, system 100 is used to distinguish between diagnoses of orthopedic conditions. For example, system 100 may distinguish between a rotator cuff issue and osteoarthritis of the shoulder. Patients that have good shoulder range of motion but that experience pain at certain positions, such as 90 degrees of elevation and internal rotation, often have a rotator cuff issue. Conversely, a patient experiencing sharp pain and restricted range of motion, such as a loss of range of motion in the external rotation and internal rotation of a shoulder, would often have an existence of osteoarthritis. Consequently, system 100, using the received 210 patient data can determine 212 a diagnosis that will assist a specialist in determining a corrective measure to aid in the recovery of the medical condition of the patient and distinguish between diagnoses of orthopedic conditions. In one embodiment, a known scoring system is used to diagnosis a condition. In this embodiment, a questionnaire associated with a known scoring system is provided to a patient and the received answers are computed based on a known scoring key.

While the examples used above were in relation to an orthopedic condition, it should be noted that system 100 and method 200 can be used in the diagnosis of any medical condition, including but not limited to, sleep disorders and eating disorders. For example, a patient having difficulty with diabetes can receive a diagnosis from system 100. Diabetics have been known to develop visual and sensory neuropathy issues such that they have trouble with a shuffling gait. After providing answers, to the appropriate questionnaire and computing device can detect the presence of a shuffling gait, such that system 100 can determine an appropriate class for the patient. Such determination can provide a patient with the information necessary to seek and obtain the appropriate care necessary to treat the medical condition.

In the exemplary embodiment, system 100 is used to track patients after a surgical procedure to determine whether a surgery is a success or has failed. After a surgery, in the exemplary embodiment, a patient utilizes method 200 at a predetermined time interval to determine the range of motion of the body portion that was affected by the surgery. Each session is stored to create a library of progression for the patient. Using this library, patients are monitored to determine if the patient is progressing, declining, or maintaining (e.g., progressively gaining or loosing range of motion over time). As such, system 100 can gauge disease severity and recommend a corrective treatment protocol.

FIG. 4 is an illustration of a patient 300 using system 100 shown in FIG. 2. In the exemplary embodiment, sensors 21 are coupled to patient 300 such that sensors 21 obtain data relating to patient 300 and transmit such data to smartphone 104. Sensors 21 coupled to an arm 302 of patient 300 are shown communicatively coupled to smartphone 104 via wires 304 and wirelessly 306. A headband 308 includes a sensor 21 embedded therein to track patient data such as but not limited to, temperature and pulse rate. In the exemplary embodiment, sensors 21 are also embedded within clothing such as shorts 310 and a shoe 312. It should be noted that any of sensors 21 can be utilized to track/monitor and transmit any data associated with patient 300. As noted above, two or more devices 104 and/or sensors 21 can be utilized together to track/monitor patient data. For example, a smartphone 104 can be used with a tablet 104 to capture different video angles of a patient to provide a 3-dimensional view. In one embodiment, processing of data received from sensors is shared and/or distributed among multiple computing devices (e.g., cloud computing). In one embodiment, sensors are ingested and/or implanted within a body.

FIG. 5 is an exemplary flowchart 400 of an entire patient experience using system 100 shown in FIG. 2. In the exemplary embodiment, system 100 is used throughout the entire patient experience. When a patient is seeking treatment, the patient will be evaluated from information obtained in patient intake 402. The patient provides information, including but not limited to, health questions, health history, and patient data received from sensors 21, in patient intake 402 via system 100. System 100 is configured to perform diagnostics 404 based on information received from a patient as described herein. In addition to performing diagnostics 404, system 100 is configured to provide treatment recommendations 406 based on input received from patients. The treatments 406 include, but are not limited to medical treatments (e.g., medicinal intervention) and surgical procedures. In the exemplary embodiment, system 100 monitors or tracks results 408 of diagnostics 404 and/or treatments 406 provided. The tracking can be done in any manner that facilitates tracking such as, but not limited to, monitoring patient movement and receiving patient data questionnaires, and/or test/lab results. Similar to monitoring or tracking results 408, system 100 is configured to monitor the progress of rehabilitation 410 a patient undergoes. Rehabilitation can be any form of rehabilitation including therapy and triage. In the exemplary embodiment, system 100 is configured to monitor, store, and compare long term results 412 of diagnostics and/or treatments 406 to evaluate the success or failure of the of diagnostics and/or treatments 406. The use of system 100 throughout an entire patient experience facilitates a higher quality of treatment with the ability to achieve high user satisfaction. It should be noted that the patient experience can apply to any medical situation including, but not limited to, orthopedic joint replacements.

In one embodiment, system 100 is configured to provide a quality of care metric based on a patient experience. In this embodiment, tracked results 408 are normalized for each patient. Normalization includes weighting results with respect to predetermined factors. The predetermined factors include, but are not limited to patient weight, patient age, and gender. Normalization can also compare results to an aggregation of patient data for patients that are similarly situated. In this embodiment, system 100 tracks and/or determines if symptoms leading to a diagnosis and/or treatment have increased, been maintained, been eliminated, and/or reduced. The quality of care metric is then determined by comparing symptomatic results to normalized results. The quality of care metric can be used by a party to determine, if and how much to pay and/or reimburse for services provided. The quality of care metric can be transmitted to and utilized by any payer including, but not limited to, a government agency and an insurance group. Additionally, tracked results and/or quality of care metrics can be used by entities to ensure compliance with regulatory requirements.

The above-described methods and systems are configured to enable telemedicine to be performed. Telemedicine can include any category of telemedicine including, but not limited to, store-and-forward, remote monitoring, and real-time interactive services. Additionally, the methods and systems can utilize any type of telemedicine including, but not limited to, telenursing, telepharmacy, telerehabilitation, teletrauma care, telecardiology, telepsychiatry, teleradiology, telepathology, teledermatology, teledentistry, teleaudiology, teleophthalmology, and telesurgery.

In one embodiment, technical effects of the methods, systems, and computer-readable media described herein include at least one of: (a) receiving input relating to a medical patient from at least one sensor; (b) determining a class based on the received input from the medical patient; and (c) transmitting the received input to a predetermined receiver associated with the medical patient, based on the determined class.

The above-described methods and systems facilitate automatic and electronic diagnosis of a medical condition of a patient. In one embodiment, patients are provided immediate feedback upon the submission of a patient data relating to a medical condition the patient is experiencing. This feedback would allow a narrowing down of diagnosis and lead to fewer diagnostic testing saving money for a patient and/or insurance carrier. The above-described methods and systems also facilitate provide appropriate medical staff with information necessary to make a more targeted treatment protocol and/or diagnosis based on the information received from the system. Because the above-described methods and systems can include patient data, the methods and systems described above can be configured to adhere to regulatory requirements, such as but not limited to the Health Insurance Portability and Accountability Act (HIPPA). In one embodiment, patient identifying information is removed from patient data.

It should be appreciated that one or more aspects of the present disclosure transform a general-purpose computing device into a special-purpose computing device when configured to perform the functions, methods, and/or processes described herein.

This written description uses examples to disclose various embodiments, which include the best mode, to enable any person skilled in the art to practice those embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for diagnosing a medical condition of a patient, said method comprising:
providing medical condition information;
receiving patient data relating to the medical condition information;
comparing the received data to a baseline; and
determining, by a computing device including a processor, a class of patient based on the received patient data.

2. A method according to claim 1, further comprising providing a directive based on the received patient data.

3. A method according to claim 2, further comprising receiving patient directive data relating to the provided directive.

4. A method according to claim 3, wherein receiving patient directive data comprises receiving video of a patient performing the provided directive.

5. A method according to claim 3, wherein receiving patient directive data comprises receiving sensor data obtained during a performance of the provided directive.

6. A method according to claim 1, wherein determining a class of patient comprises diagnosing a medical condition.

7. A method according to claim 1, wherein providing medical condition information comprises providing a medical condition questionnaire.

8. A method according to claim 1, wherein determining a class of patient comprises determining, by a portable computing device, a class of patient.

9. One or more non-transitory computer-readable storage media having computer-executable instructions embodied thereon are provided, wherein when executed by a processor, the computer-executable instructions cause the processor to:
provide medical condition information;
receive patient data relating to the medical condition information;
compare the received data to a baseline; and
determine a class of patient based on the received patient data.

10. One or more non-transitory computer-readable storage media according to claim 9, wherein the computer-executable instructions cause the processor to provide a directive based on the received patient data.

11. One or more non-transitory computer-readable storage media according to claim 10, wherein the computer-executable instructions cause the processor to receive patient directive data relating to the provided directive.

12. A method for determining a quality of care score for treatment associated with a patient, said method comprising:
receiving patient data;
tracking at least one treatment provided to a patient;
monitoring at least one result of the at least one treatment; and
determining, by a computing device including a processor, a quality of care score based on the at least one result.

13. A method according to claim 12, wherein determining a quality of care score comprises comparing the at least one result to the received patient data.

14. A method according to claim 12, wherein determining a quality of care score comprises comparing the at least one result to a baseline.

15. A method according to claim 12, further comprising normalizing the at least one result.

16. A method according to claim 12, further comprising transmitting the determined quality of care score to a payer of the at least one treatment provided.

17. A method according to claim 12, further comprising determining at least one of a payment amount and a payment percentage based on the determined quality of care score.

18. One or more non-transitory computer-readable storage media having computer-executable instructions embodied thereon are provided, wherein when executed by a processor, the computer-executable instructions cause the processor to:
receive patient data;
track at least one treatment provided to a patient;
monitor at least one result of the at least one treatment; and
determine a quality of care score based on the at least one result.

19. One or more non-transitory computer-readable storage media according to claim 18, wherein the computer-executable instructions cause the processor to normalize the at least one result.

20. One or more non-transitory computer-readable storage media according to claim 18, wherein the computer-executable instructions cause the processor to transmit the determined quality of care score to a payer of the at least one treatment provided.
